(19) United States
(12) Patent Application Publication
(10) Pub. No.: US 2002/0097388 A1
(43) Pub. Date: Jul. 25, 2002

(54) Multi-spectral imaging system and method for cytology

(76) Inventor: Rayn S. Raz, Toronto (CA)

Correspondence Address: HARNESS, DICKEY & PIERCE, P.L.C., P.O. BOX 8910, RESTON, VA, 20195, US

(21) Appl. No.: 10/098,374

(22) Filed: Mar. 18, 2002

Related U.S. Application Data
(63) Continuation of application No. 09/430,012, filed on Oct. 29, 1999.

(30) Foreign Application Priority Data
May 9, 1997 (CA) PCT/CA97/00318

(51) Int. Cl. G01N 33/48

(52) U.S. Cl. 356/39

(57) ABSTRACT

A multi-spectral image system and method for cytology. The multi-spectral imaging system comprises an optical stage, an image capture camera, and a controller. The optical stage includes a light source for illuminating the cytological specimen and optical means for producing images of the illuminated specimen in a number of spectral bands. The image capture camera includes means for simultaneously capturing the spectral images and generating electrical signals corresponding to the captured images. The controller controls the operation of the image capture camera and the light source and includes means for converting the electrical signals into a data form suitable for further processing. The multi-spectral imaging system is particularly suited for specimens prepared in the form of thin-layers or monolayers. The image data produced by the relevant state of the specimen and also permits the use of human-expert review for confirmation.

### FIELD OF THE INVENTION

The present invention relates to automated biological testing systems and more particularly to a system for generating data for the analysis of the visual characteristics of cytological specimens, and in particular biological specimens obtained for Papanicolaou (Pap) testing and prepared as a monolayer specimen.

### BACKGROUND OF THE INVENTION

In the art, there are known techniques for the machine-aided evaluation of biological or medical specimens. Many of these embody the application of optical decomposition for image evaluation.

Bacus, in U.S. Pat. No. 5,202,931, teaches an optical method and apparatus for protein quantification that utilizes two band-pass optical filters centred at 500 nm and 650 nm. The filters are optimized to produce maximal contrast between cellular nuclei with and without diaminobenzidine precipitate staining. While the Bacus invention is effective for application in a quantitative immunohistochemical assay, the Bacus method is not suitable to capture and exploit the crucial properties of a Papanicolaou (Pap) test for automated evaluation. Specifically, the Pap test evaluation does not reduce to a simple binary decision, i.e. either a “yes” or a “no” for the presence of a specific staining precipitate. The Pap test evaluation requires the synthesis of a highly-variable and wide-ranging set of visual and clinical circumstances in order to render a diagnostically reliable outcome. From the perspective of machine automation, these visual circumstances are the complete range of mathematical “features” which are raised as a consequence of the standardized staining protocol. Thus, any application of the image analysis techniques to the Pap test must be constrained to this stain and must extract the full range of features that replicate the appreciation gained through human visual evaluation.

In U.S. Pat. No. 4,191,940, Polcyn et al. discloses a technique for the use of a decomposed set of optical wavelengths for a multivariate analysis of cell identification. Though powerful in its own right, the Polcyn technique is limited to the separation of different categories of material based on simple absorption properties alone. As described above, the Pap test is much more subtle and complex. The optical absorption properties represent only the beginning of the chain of analysis that ultimately leads to a medical diagnosis. Given the complexity of the cervical cytology application it is usual to apply what is known as a “classical” image analysis consisting of segmentation, feature extraction and classification. In this way only is it possible to arrive at a precise and accurate classification of the myriad components that reside within a gynaecological specimen.

The complexity of the Pap test automation task is borne out in U.S. Pat. No. 5,287,272 by Rutenberg et al. Rutenberg et al. teaches a method and apparatus that draws a clear distinction between the conventional Pap smear and the thin layer or monolayer specimens that are the subject of the present invention. According to Rutenberg et al., the application of cytological image analysis is severely constrained by limitations the conventional Pap smear. Unlike the controlled monolayer specimen, the conventional smear is characterized by irregular cell groupings and distributions, thick, overlying cell clusters and occluding debris. By avoiding the monolayer preparation, Rutenberg et al. are restricted to a level of image analysis that is limited in its sensitivity and specificity.

The subject invention addresses the problems and limitations associated with the prior art. The present invention utilizes a monolayer specimen for automated cytological analysis and advantageously features a segmentation phase with improved accuracy and produces a complex and extensive range of extracted features. This allows a more refined approach to the problem of cytological classification and improves performance and provides cost savings. The image collection component of this invention also features the creation of a “pseudo-coloured” image that retains the bulk of the visual cues required by cyto-technologists for interactive review purposes.

Constrained by the nature of the preparation, the fixed protocol of the biological staining and the necessity to bridge the gap between machine processing and human evaluation, the present invention comprises a refined set of optical filters used in conjunction with a high-speed imaging system, processing hardware, discriminant-analysis techniques and mathematical measures to pre-process images for cellular identification. The images gathered generated according to the invention are also useful for human-interactive review, a further advantage of the system.

### BRIEF SUMMARY OF THE INVENTION

The present invention provides an imaging system having the capability to simultaneously capture the same scene in multiple spectral bands, and comprises a system having an integrated optical system, image collection devices and a method for pre-processing and analyzing human cervical cytology specimens or samples. The system is particularly suited for specimens prepared in the form of thin-layers or monolayers. The image data produced by the system is suitable for automated eassessment of the clinically-relevant state of the specimen and also permits the use of human-expert review for confirmation or to establish diagnostic grade and clinical action.

The system according to the present invention comprises three principal components (a) optical hardware (b) electronic hardware and (c) measurement and analysis procedures and methods. The optical hardware provides for illumination of the specimen, magnifies the cellular components, separates the appropriate wavelengths and directs the separated wavelengths for electronic digitization. The electronic hardware provides for the translation of the optical images into digital information and for the overall control of the processing steps according to the invention. The measurement and analysis procedures preferably comprise processing steps embedded in hardware for pre-processing the information for classification.

This subject invention is intended to function with components described in co-pending patent applications entitled Automated Scanning of Microscope Slides International Patent Application No. CA96/00475 filed Jul. 18, 1996 and U.S. Pat. Application No. 60/001,220 filed Jul. 19, 1995, Pipeline Processor for Medical and Biological Applications U.S. patent application Ser. No. 08/683,440 filed Jul. 18, 1996 and U.S. Patent Application No. 60/001,219 filed Jul. 19, 1995, Multi-Spectral Segmentation International Patent Application No. CA96/00477 filed Jul. 18, 1996 and U.S. Patent Application No. 60/001,221 filed Jul. 19, 1995, Neural-Network Assisted Multi-Spectral Segmentation International Patent Application No. CA96/00619 filed Sep. 18, 1996 and U.S. Patent Application No. 60/003,964 filed Sep. 19, 1995, Automated Focus System International Patent Application No. CA96/00476 filed Jul. 18, 1996 and Window Texture Extraction International Patent Application No. CA96/00478 filed Jul. 18, 1996 and U.S. Patent Application No. 60/001,216 filed Jul. 19, 1995, all in the name of the common owner.

In a first aspect, the present invention provides an imaging system for capturing multi-spectral image data of a cytological specimen, said imaging system comprising: (a) an optical stage having a light source for illuminating the specimen, and optical means for producing images of the illuminated specimen in a plurality of spectral bands; (b) an image capture camera having means for simultaneously capturing said spectral images and generating corresponding electrical signals corresponding to said captured spectral images; (c) controller means for controlling the operation of said image capture camera and said light source, said controller means having means for converting said electrical signals corresponding to said captured spectral images into a data form suitable for further processing.

In another aspect, the present invention provides a method for generating multi-spectral image data for cytological specimen, said method comprising the steps of: (a) exposing said cytological specimen to a short burst of broad-band light; (b) separating said burst of broad-band light into a plurality of spectral bands; (c) simultaneously capturing an image for each of said spectral bands and generating electrical signals corresponding to each of said captured spectral images; (d) converting the electrical signals corresponding to said captured spectral images into a data form suitable for further processing.

### BRIEF DESCRIPTION OF THE DRAWINGS

Reference will now be made, by way of example, to the accompanying drawings which show preferred embodiments of the present invention, and in which:

FIG. 1 shows in block diagram form a multi-spectral imaging system according to the present invention;

FIG. 2 shows in a diagrammatic form an optical pathway for the multi-spectral imaging system of FIG. 1;

FIG. 3 shows spectral bands for images captured;

FIG. 4 shows in block diagram form an electronic circuit for the multi-spectral imaging system according to the present invention; and

FIG. 5 shows in block diagram a camera for the multi-spectral imaging system according to the present invention.

### DETAILED DESCRIPTION OF THE PREFERRED EMBODIMENTS

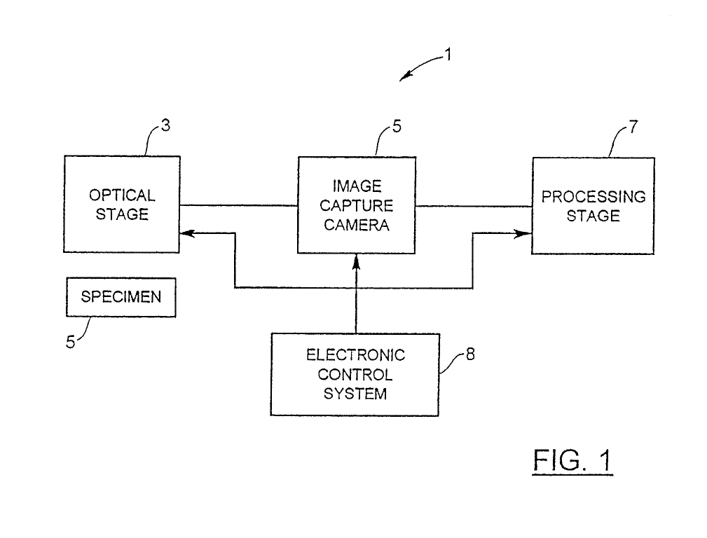

Reference is first made to FIG. 1 which shows in block diagram form a multi-spectral imaging system 1 according to the present invention. The multi-spectral imaging system 1 comprises an optical stage 3, an image capture camera 5, and a processing stage 7 and an electronic control system 8.

As will be described, the multi-spectral imaging system 1 provides a method and apparatus for generating data representing the visual characteristics of a cytological specimen denoted by reference S in FIG. 1. According to one aspect of the invention, the data is generated in a form which facilitates further processing and analysis of the characteristics of the cytological specimen S and is particularly suited for monolayer specimens.

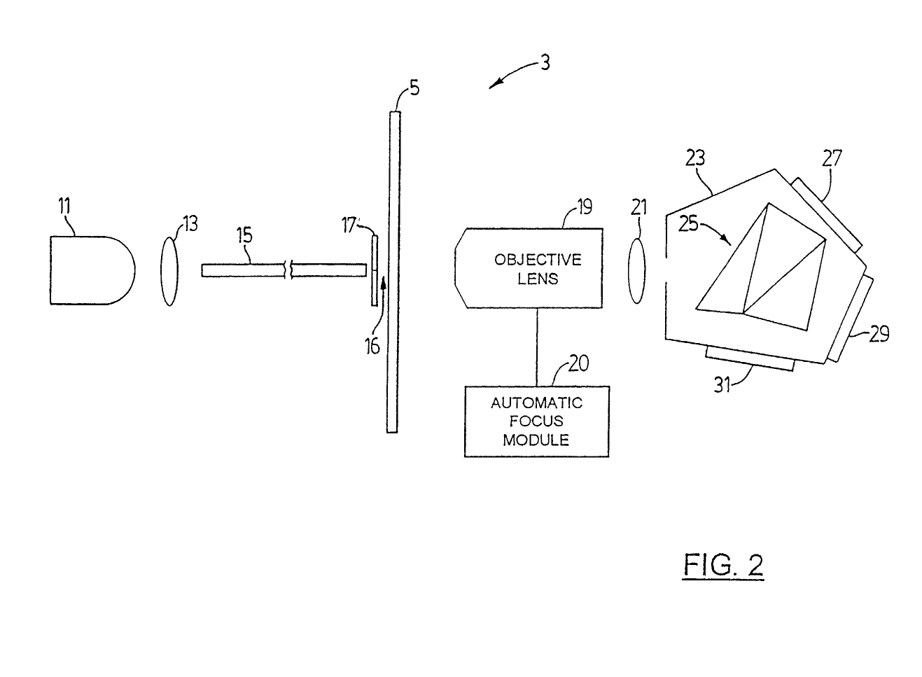

Reference is made to FIG. 2 which shows the optical stage 3 in more detail. The optical stage 3 provides the optical path for the system 1. The optical stage 3 includes a high-intensity electrical discharge tube 11, a condensing lens 13, a fibre-optic bundle 15, a small aperture 17, an objection lens 19, a telan lens 21, and a prism assembly 23. The prism assembly 23 includes an optical element 25 with filters 27, 29, 31.

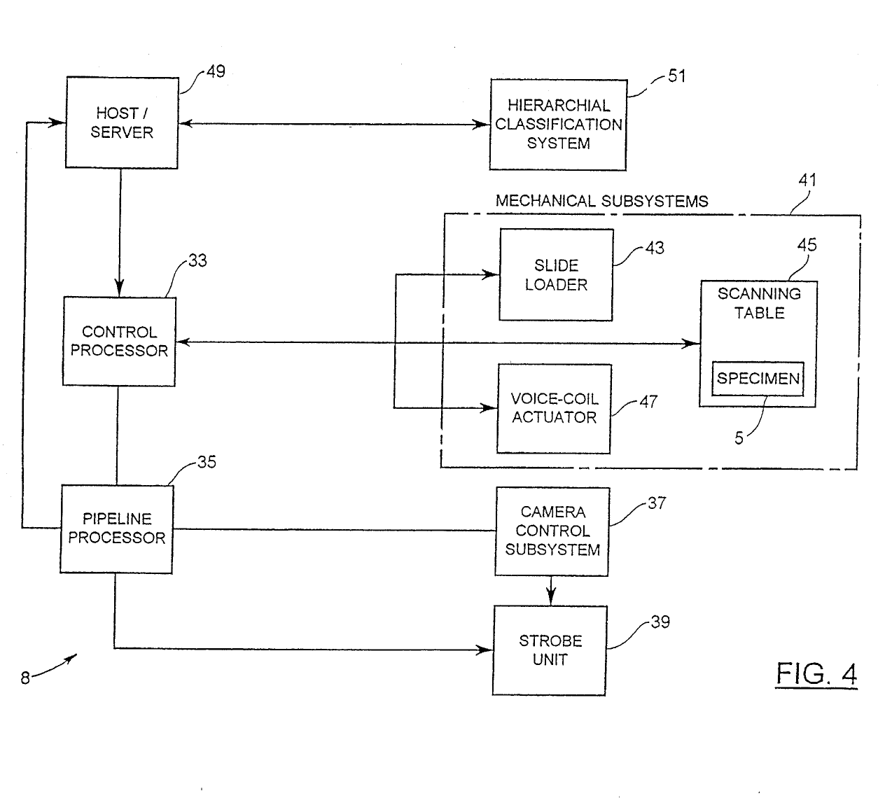

The electrical discharge tube 11 is operated as a stroboscopic lamp. Preferably, the discharge tube 11 produces a short intense pulse of light lasting less than 6 microseconds. The lamp 11 is selected to have a broad-band spectral output covering a range between 400 nm and 700 nm. As will be described, the optical filters 27, 29, 31 select the appropriate wavelengths for image formation from this broad range. The pulse of light must have sufficient intensity to accommodate losses from the intervening optics. A short light pulse is preferred because it allows the multi-spectral system 1: (a) to isolate from the image mechanical vibrations that result in mechanical velocities of less than 0.08 meters per second at the microscope slide level, (b) to operate the CCD array cameras (see FIG. 4 below) without electronic or mechanical shutters thereby increasing the rate of image acquisition, and (c) to illuminate the sample without the photo-bleaching or heat damage effects associated with continuous illumination sources.

The light emitted by the strobe lamp 11 is coupled to the fibre-optic bundle 15 by the condensing lens 13. The condensing lens 13 comprises a known optical element which functions to gather, concentrate, collimate and project the light emitted by the strobe lamp 11 onto the face of a fibre-optic bundle 15. The fibre-optic bundle 15 preferably comprises a tightly-packed group of glass fibre-optic cables. The primary function of the fibre-optic bundle 15 is to couple the light from the lamp 11 to illuminate the specimen S. The use of a fibre-optic bundle 15 as a light guide is preferred because it allows the strobe lamp 11 to be operated at some distance from the object plane, i.e. specimen S, of the system 1. Advantageously, this arrangement reduces the potential occurrence of electrical interference from the intense electrical discharges occurring at the lamp 11. The flexibility of the fibre-optic bundle 15 also permits the use of indirect optical paths from the strobe lamp 11 to the object plane and thereby eases design considerations.

As shown in FIG. 2, the small aperture 17 is centred on the optical axis of the objective lens 19 at the exit face of the fibre-optic bundle 15. This arrangement is preferred because it restricts the illumination to the region immediately surrounding the region of interest (denoted by 16 in FIG. 2) and advantageously reduces the contrast-reduction effects associated with internal reflections within the optical components and yields better-resolved images.

The light which passes through the specimen S is collected by an objective lens 19. The objective lens 19 preferably comprises an infinite-conjugate optical system. The objective lens 19 preferably has moderate nominal magnification (×10 or ×20) and a numerical aperture of 0.4 NA-0.75 NA. The lens 19 is brought into the correct or optimal focus for the nuclear material contained in the specimen S within the field of view by means of an automatic focus module 20. The automatic focus module 20 is preferably implemented as the apparatus and method as substantially described in co-pending PCT Patent Application No. CA96/00476 filed in the name of the common owner. The automatic focus techniques which control the focus mechanism are used in conjunction with a method of image formation by spectral separation as will be described below in further detail. As described in co-pending International Patent Application No. CA96/00476 (which is hereby incorporated by reference) the automatic focus module 20 comprises a servo-mechanical mechanism having a magnetically-suspended voice-coil actuator 47 (FIG. 4) which supports the objective lens 19. The voice-coil actuator 47 receives motion control instructions from the electronic control system 8 based upon the mathematical calculations and process control steps as described in the co-pending application for an automated focus system.

The objective lens 19 preferably comprises an infinite-conjugate objective lens which produces a real image of the specimen S that is projected (theoretically) to an infinite distance. In the optical stage 3 the light emitted from the infinite-conjugate lens 19 is subsequently gathered by the telan lens 21. The function of the telan lens 21 is to create and project a real image to a finite position within the prism assembly 23. An infinite-conjugate system is preferred for the following reasons. First, the magnification is a function only of the ratio of the focal length of the objective lens 19 and the telan lens 21. This means that the magnification is not sensitive to the relative displacement of the objective lens 19 and so the motion of the objective lens 19 during the automatic focusing will have negligible effect upon the optical magnification of the system 1. This is in contrast to a conventional DIN microscope system in which the magnification is based on a specific tube length (e.g. 160 mm with 45 mm parfocal length). A second advantage of the present arrangement is that the light between the objective lens 19 and the telan lens 21 is collimated. Thus, it is possible to introduce additional optical elements, such as beam-splitters, without suffering or incurring spherical aberrations in the final image. Thirdly, the infinite-conjugate objective lens 19 allows the simple alteration of the magnification of the real image by a substitution of an objective lens of a different focal length. Unlike conventional finite tube length systems, the alteration of the arrangement shown in FIG. 2 would carry no penalty with respect to the quality of the image obtained from the specimen S.

The image re-formed by the telan lens 21 is projected into the prism assembly 23. The prism assembly 23 comprises the internal optical prism element 25 with the three optical filters 27, 29, 31 which are optically coupled to respective faces of the prism assembly 23. The function of the prism assembly 25 is to select a series of three narrow optical wavelength representations of the image. The three optical wavelengths are based in part on spectral decomposition principles as described by G. Coli et al. in Olivetti Research and Technology Review Vol. 8, No. 33 (1987).

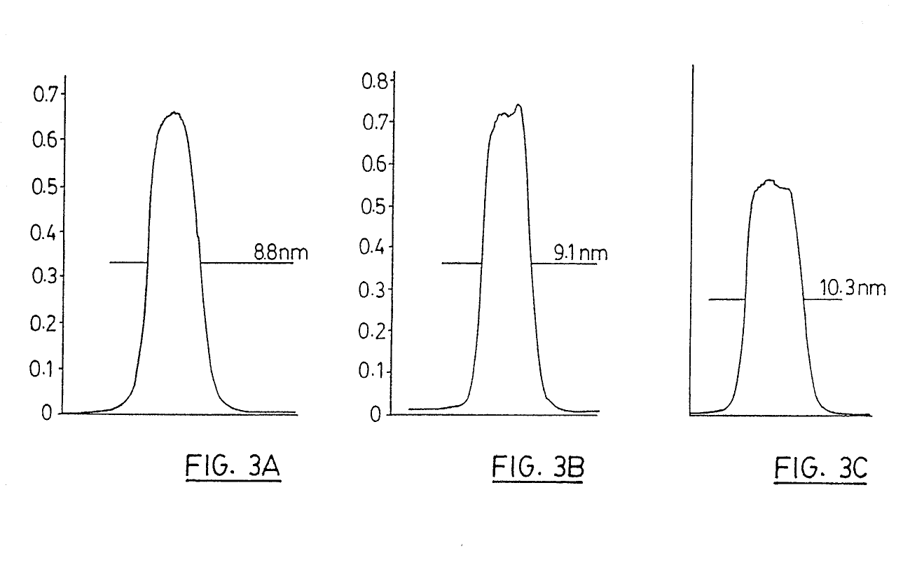

The optical prism element 25 comprises a set of glass wedges coated with dielectric film stacks to create the interference band-pass optical filters 27, 29, 31. By selecting wedge angles and dielectric film coatings the prism 23 will simultaneously produce three images from the same scene in each of three narrow optical regions. The width of each of these optical regions is preferably 10 nm with a transmission efficiency of at least 50% within the optical band. The three centre wavelengths for these bands are selected as 530 nm (I), 577 nm (II) and 630 nm (III) as shown in FIG. 3.

The arrangement according to this aspect of the invention has specific advantages for the acquisition and processing of images derived from Papanicolaou-stained human epithelial cells, such as those encountered in the Pap test. The prism assembly 23 features a compact and robust design with very high natural vibration frequencies. Thus the prism assembly 23 is immune from the much lower frequencies that typify ambient mechanical vibrations. Once assembled and aligned, the prism assembly 23 is highly stable against thermal or mechanical drift and as such reduces additional servicing over its useful lifetime.

In another aspect, the prism simultaneously produces three spectrally-selective images thus conferring a factor of three reduction in the acquisition time for images needed in the processing stages. In addition, the simultaneous capture is advantageous because it reduces the number of strobe flashes required of the lamp 11 by a factor of three. This, in turn, increases the operating life of the lamp 11 and also the lifetime of the stains that are present in the specimen S itself. The simultaneous image acquisition feature also reduces the possibility of image mis-alignment among the three images due to vibrations.

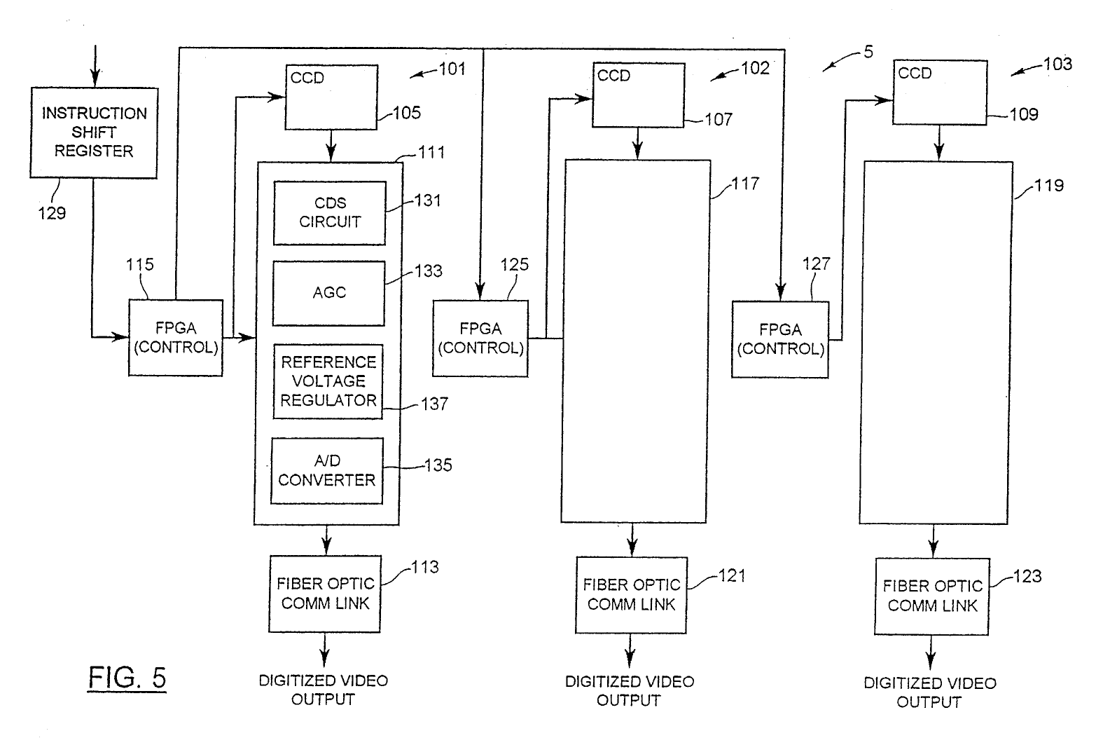

The three spectrally-selected images produced by the optical stage 3 are fed to the image capture camera 5 (FIG. 1). The image capture camera 5 comprises a CCD (Charge Coupled Device) camera which digitizes each of the three spectral images. The image capture camera 5 is described in greater detail below with reference to FIG. 5. The acquisition, digitization, storage and pre-processing of the three spectrally-selected images is controlled by an electronic control system 8 as shown in FIG. 4.

Reference is made to FIG. 4 which shows in block diagram the electronic control system 8 for the multi-spectral imaging system 1. The electronic control system 8 comprises a control processor 33, a pipeline processor 35, a camera control subsystem 37, and a strobe unit 39. As shown in FIG. 4, the control processor 33 provides an interface to the mechanical subsystems 41. The mechanical subsystems 41 comprise a slide loader 43, a scanning table 45 and the voice-coil actuator 47. Elements of the electronic control system 8 and the mechanical subsystems 41 are subjects of co-pending patent applications filed in the name of the common owner and referenced by International Patent Application No. CA96/00476 entitled Automatic Focus System, International Patent Application No. CA96/00475 entitled Spiral Scanner for Microscope Slides, and U.S. patent application Ser. No. 08/683,440 entitled Pipeline Processor for Medical/Biological Image Analysis.

Normal operation of the multi-spectral imaging system 1 is initiated by a call or request to the electronic control system 8. The request is typically issued by a host/server 49 for image data and/or mathematical feature data which is derived from a captured image.

The request from the host/server is directed to the control processor 33 which is responsible for the overall control of the image acquisition systems comprising the camera 37, strobe unit 39 and mechanical subsystems 41. According to this aspect of the invention, the control processor 33 is suitably programmed to synchronize and integrate the operations of the mechanical subsystems 41, camera control subsystem 37 and the pre-processing or pipeline processor 35 so as to comply and complete the request of the host/server.

In operation, the control processor 33 first determines the state of the slide loader 43 and scanning table 45. (The operation of a preferred slide loader is described in co-pending PCT Patent Application No. CA96/00475 and U.S. Patent Application No. 60/001,220, and the operation of a preferred voice-coil actuator for an automatic focusing system is described in co-pending PCT Patent Application No. CA96/00476 and U.S. Patent Application No. 60/001,218.) The control processor 33 determines whether a slide carrying the specimen S is present in the scanning table 45 or whether a slide is being loaded or unloaded. The control processor 33 also receives signals with respect to the precise position of the slide on the scanning table 45 in relation to the optical axis of the system through a rotary encoding system (not shown). The control processor 33 then issues instructions to the voice-coil actuator 47 based on information provided by the pipeline processor 35 with respect into optimal focus position.

When the mechanical subsystems have been appropriately positioned, the control processor 33 instructs the camera subsystem 37 and the pipeline processor 35. The camera subsystem 37 initiates capture of an image, and the captured image is then pre-processed by the pipeline processor 35 and the data generated is sent to the host/server 49. For these functions, control preferably devolves to the local level of the control CPU in the pipeline processor 35 which is responsible for the image data requests and the pre-processing timing and synchronization.

The control CPU in the pipeline processor 35 determines the availability of memory, the timing conditions for the pipeline processor 35 and the status of the camera subsystem 37. If the camera 37 and mechanical subsystems 41 are ready, the control CPU initiates a stroboscopic flash by means of a trigger command to the strobe unit 39. Histogram processing in the pipeline processor 35 determines if the strobe unit 39 must adjust its intensity, and if necessary an analog signal is sent to the strobe unit 39 for such an adjustment before the flash is initiated. After the light pulse from the strobe lamp 11 is completed, the camera subsystem 37 converts the light signal into digital information.

According to this aspect, the camera subsystem 37 simultaneously digitizes the three images produced by the optical stage 3 (FIG. 2). After the digitization of the three spectrally-resolved images, all three digitized images are simultaneously transmitted from the camera subsystem 37 to the input stage of the pipeline processor 35 over three separate fibre-optic links (FIG. 5).

The pipeline processor 35, under the control of the control processor 33, performs the pre-processing steps required before classification procedures can be applied to the digitized images. The pre-processing operations include one of two types of segmentation procedures: (i) a multi-spectral segmentation operation, or (ii) a neural-network assisted multi-spectral segmentation operation. The multi-spectral segmentation process is described in co-pending PCT Application No. CA96/00477 and U.S. Patent Application No. 60/001,221, and the neural-network assisted multi-spectral segmentation process is described in copending PCT Application No. CA96/00619 and U.S. Patent Application No. 60/003,964. The pipeline processor is described in co-pending U.S. patent application Ser. No. 08/683,440 and U.S. Patent Application No. 60/001,219. The segmentation operation is followed by an extraction operation wherein a wide range of features from the segmented objects within the digitized images are extracted. The pipeline processor 35 is also responsible for image levelling routines, focus number calculations and histogram recalculations. The histogram calculations are used for proper light intensity control. When the segmentation and feature extraction operations are complete, the pipeline processor 35 sends the features to the host/server 49 along with the images (if requested by the host/server 49). The processed features are then. fed into a hierarchical classification system 51. The principal function of the hierarchical classification system is to make decisions regarding the identity of the segmented objects, such as, identifying features or characteristics in the nuclei of cervical cells corresponding to medical prognosis.

As described above, a feature of the present invention is the simultaneous capture of three spectrally-resolved images of cellular matter and the subsequent digitization and processing of the image data. The image capture camera 5 is controlled by the camera control subsystem 37 (FIG. 4) as described above. The image capture camera 5 according to this aspect of the invention is shown in more detail in FIG. 5. The primary function of the image capture camera 5 is the digitization of the images for processing and analysis. Referring to FIG. 5, the image capture camera 5 comprises three image processing stages 101, 102, 103, one for each spectral band. Each of the image processing stages 101, 102, 103 includes a Charge Coupled Device (CCD) array 105, 107, 109. The first image processing stage 101 comprises the CCD array 105, an analog-to-digital interface module 111, and optic communication link 113. The image processing stage 101 is controlled by signals generated by a control module 115. Similarly, the second and third image processing stages 102, 103 comprise respective analog-to-digital interface modules 117, 119, fibre-optic communication links 121, 123 and control modules 125, 27. The Charge Coupled Device (CCD) arrays 105, 107, 109 are utilized for capturing three spectrally-resolved images. Charge Coupled Devices are preferred because they are stable, solid-state elements which have a linear response to visible light over a wide spectral range. The CCD arrays 105, 107, 109 provides a high rate of image capture in a digital format that is particularly suited to computer processing and display. Advantageously, the CCD arrays 105, 107, 109 permit the imaging system 1 to avoid complications associated with analogue cameras such as baseline drift, re-sampling errors and analogue noise. The CCD arrays 105, 107, 109 take the form of area (rather than linear) scan arrays of 512 vertical by 768 horizontal picture elements (“pixels”). By employing accurate timing of the scan lines, the images drawn from the CCD arrays utilize only 512 of the 768 pixels available in the horizontal dimension. This allows a shift of image position by up to 50% without the need to resort to mechanical adjustments.

According to the invention, the images of the cervical cells are simultaneously examined by three narrow (10 nm) interference band-pass filters 27, 29, 31 (FIG. 2). This allows a maximization of the image contrast between the nucleus and the cytoplasm in the specimen S and between the cytoplasm and the background.

The CCD arrays. 105, 107, 109 used in the image capture camera 5 preferably comprise the CCD array manufactured by Kodak under model number KAF-0400. The KAF-0400 model CCD array is a full-frame image sensor, i.e. the CCD device captures and transfers an entire video frame rather than using alternating image “fields” composed of odd and even rows (known in the art as the interline transfer technique). The use of a full-frame sensor is preferred because it simplifies the electronics while maintaining image resolution. The maximum data rate for the KAF0400 model CCD array device is 20 MHz which allows a theoretical image capture limit of 40 frames/sec. The picture elements of the CCD array are square (9 microns×9 microns). This feature eliminates the need for the aspect-ratio corrections as required in television receivers for example. In addition, the CCD array provides a 100% fill factor for the pixels. This means that a negligible amount of light is lost to the depletion regions that confine the photo-generated electrons to each individual pixel. The KAF-0400 CCD array does not have an electronic “shutter” which allows it to clear out and reset all the pixels between capturing and transferring images. However, as the illumination system consists of an arc-discharge strobe lamp 11 the integration of stray light between images does not pose a problem. In another aspect, each “line” of the CCD array 105, 107, 109 has a number of “black” reference level pixels that are completely shielded from light. The “black” pixels are measured to establish a baseline for the CCD array on a line-by-line basis. This allows an immediate adjustment for drifts in sensitivity due to temperature or electrical fluctuations in the CCD array.

Referring to FIG. 5, each CCD array 105, 107, 109 is coupled to the respective control module comprising a Field-Programmable Gate-Array (FPGA) 115, 125, 127. The first FPGA 115 is also coupled to a command register 129. The command register 129 comprises a shift register which receives instructions from an external source, in this case, the command register 129 receives control commands from the control CPU in the pipeline processor 35. The commands issued by the pipeline processor 35 instruct the FPGA 115 to “take a picture”. The other two FPGA's 125, 127 are coupled to the first FPGA 115 through a “daisy-chain” and also receive the command. The FPGA's 125, 127, 115 comprise digital logic circuits and are configured to issue control signals in response to commands received from the control CPU in the pipeline processor 35 for controlling the operation of the respective image processing/capture stage 101, 102, 103. In particular, each FPGA 115, 125, 127 is programmed to synchronize the respective CCD array 105, 107, 109 and initiate the timing procedures for capturing and digitizing each of the spectrally-resolved images. In operation, each FPGA 115, 125, 127 synchronizes the respective CCD array 105, 107, 109 and initiates the timing procedures. The first FPGA 115 then sends a signal via the interface register 129 and pipeline processor 35 to the strobe unit 39 to initiate a flash and then the capture of the three spectrally-resolved images. After the flash is complete, the transfer and pre-processing of image data from the three CCD arrays 105, 107, 109 is commenced simultaneously.

Referring to FIG. 5, the contents of each pixel in the CCD array 105, 107, 109 are shifted out one-by-one to the respective analog-to-digital interface module 111, 117, 119. The analog-to-digital interface modules 111, 117, 119 are preferably implemented using the single-channel analog-to-digital signal interface available from Philips Semiconductors under model number TDA-8786. The TDA-8786 analog-to-digital interface features a Correlated Double Sampling (CDS) circuit 131, automatic gain control (AGC) 133, a 10-bit analog-to-digital converter 135, a reference voltage regulator 137, and is fully programmable via a serial interface, as will be understood by one skilled in the art.

As shown in FIG. 5, the analog-to-digital interface modules accept and measure the electronic charge from the CCD camera arrays 105, 107, 109 using the internal correlated double sampling circuitry 131. The output voltage is amplified within the analog-to-digital interface through an internal voltage-controlled voltage amplifier 133. The gain of this voltage controlled voltage amplifier 133 is controlled by an on-chip digital-to-analog converter (not shown) that receives instructions via a serial interface coupled to the FPGA 115, 125, 127. This arrangement allows the FPGA 115, 125, 127 to electronically adjust the gain of the video signal produced by the respective CCD array 105, 107, 109.

The “optical black clamp” in the analog-to-digital interface 111, 117, 119 is timed to sense the output of the first “black” pixels mentioned above. The voltage values extracted from the “black” pixels are used to off-set the sample-and-hold circuit so as to compensate for drifts in the response of the CCD array 105, 107, 109 in a line-by-line fashion.

The output signals from the CCD arrays 105, 107, 109, now converted to voltage values, are sent to the on-board analog-to-digital converter 135. The analog-to-digital converter 135 is capable of 10 bits accuracy, but as will be understood by one skilled in the art the usable output will be limited by the bandwidth of the analog video signal received from the video differencing amplifiers 133 contained within the analog-to-digital signal interfaces 111, 117, 119.

The digital video signal derived from the output for each CCD array 105, 107, 109 is transmitted via the respective fibre-optic link 113, 121, 123 to the computational sections of the pipeline processor 35.

As described above, a feature of the multi-spectral imaging system 1 is the capability to simultaneously capture the same scene in each of three narrow optical bands, 530 nm, 577 nm and 630 nm.

The use of the spectrally-resolved images according to the present invention as described above permits a more refined and accurate measure of the relevant biological characteristics of the segmented objects such as DNA quantification, etc. In this aspect, the multi-spectral imaging technique both concentrates attention on the relevant biological measures and greatly multiplies the number of features available for the classification stage. This is an important advantage because it is usually not known at the outset which, if any, features will be of value to classification. Additional applications and techniques for feature extraction with these spectrally-resolved images may be found in the co-pending PCT Patent Application No. CA96/00478 for a Window Texture Extraction method. Another advantage of the multi-spectral imaging system is the reduction in the sensitivity to stain variations. The use of these three narrow optical bands reduces the sensitivity of the classification to variations in the quality and intensity of the Papanicolaou stain. The application of this stain protocol is very much site-dependent, and variations are typically only noticed when they begin to interfere with the human interpretation of the Pap tests. If an automated analysis system is to be commercially-viable then it must not be over-sensitive to these stain variations. The use of the three narrow optical bands allows the contraction of a set of stain-invariant, or at the very least, less stain-sensitive features based on the ratios of the three optical bands. This improves the versatility of the classification system and advantageously its commercial value.

The present invention may be embodied in other specific forms without departing from the spirit or essential characteristics thereof. Therefore, the presently discussed embodiments are considered to be illustrative and not restrictive, the scope of the invention being indicated by the appended claims rather than the foregoing description, and all changes which come within the meaning and range of equivalency of the claims are therefore intended to be embraced therein.

1. An imaging system for capturing multi-spectral image data of a cytological specimen, said imaging system comprising:
(a) an optical stage having a light source for illuminating the specimen, and optical means for simultaneously producing images of the illuminated specimen in a plurality of spectral bands;
(b) an image capture means for simultaneously capturing said spectral images; and
(c) processing means for processing said captured spectral images simultaneously to evaluate the illuminated specimen.

2. The imaging system as claimed in claim 1, wherein said cytological specimen comprises a monolayer specimen.

3. The imaging system as claimed in claim 1, wherein said optical means comprises a prism assembly, said prism assembly being optically coupled to the output of said light source and having an optical element for producing each of said spectral images.

4. The imaging system as claimed in claim 3, wherein said prism assembly includes a narrow band optical filter for each of said spectral bands.

5. The imaging system as claimed in claim 4, wherein said spectral bands comprise a first optical band centered at 530 nanometers and having a width of approximately 10 nanometers, a second optical band centered at 630 nanometers and having a width of approximately 10 nanometers, and a third optical band centered at 577 nanometers and having a width of approximately 10 nanometers.

6. The imaging system as claimed in claim 1, wherein said light source comprises a broad-band strobe lamp having means responsive to a control signal received from said processing means for illuminating the specimen for a predetermined time.

7. The imaging system as claimed in claim 1, wherein said image capture means comprises a charge coupled device (CCD) for each of the spectral bands and said processing means includes an analog processor coupled to the output of each of said CCDs for generating electrical signals corresponding to each of said captured spectral images.

8. The imaging system as claimed in claim 7, wherein said processing means comprises analog-to-digital converters for simultaneously digitizing said captured spectral images.

9. The imaging system as claimed in claim 8, wherein said processing means further includes an amplifier coupled to the output of each of the analog processors and the input of the respective analog-to-digital converter.

10. The imaging system as claimed in claim 7, wherein said processing means includes a high speed communication link for each of said spectral bands for transferring said digitized captured spectral images.

11. The imaging system as claimed in claim 1, wherein said processing means comprises a dedicated hardware encoded controller module for each of the spectral bands, and includes an interface register coupled to said controller modules for receiving command information.

12. The imaging system as claimed in claim 1, wherein the light source is a stroboscopic lamp.

13. The imaging system as claimed in claim 1, wherein the processing means performs a multi-spectral segmentation on said captured spectral images.

14. The imaging system as claimed in claim 13, wherein the processing means performs a feature extraction operation on output of the segmentation operation.

15. The imaging system as claimed in claim 14, wherein the processing means performs a classification operation on output of the extraction operation.

16. The imaging system as claimed in claim 1, wherein the processing means simultaneously digitizes said captured spectral images.

17. The imaging system as claimed in claim 1, wherein the processing means controls operation of said image capture camera and said light source.

18. A method for generating multi-spectral image data for a cytological specimen, said method comprising the steps of:
(a) exposing said cytological specimen to a short burst of broad-band light;
(b) separating said burst of broad-band light into a plurality of spectral bands;
(c) simultaneously capturing an image for each of said spectral bands; and
(d) processing said captured spectral images simultaneously for evaluating the illuminated specimen.

19. The method as claimed in claim 18, wherein said cytological specimen comprises a monolayer specimen.

20. The method as claimed in claim 18, wherein said spectral bands comprise a first optical band centered at 530 nanometers and having a width of approximately 10 nanometers, a second optical band centered at 630 nanometers and having a width of approximately 10 nanometers, and a third optical band centered at 577 nanometers and having a width of approximately 10 nanometers.

21. The imaging system as claimed in claim 18, wherein the processing step performs a multi-spectral segmentation on said captured spectral images.

22. The imaging system as claimed in claim 18, wherein the processing step simultaneously digitizes said captured spectral images.

23. An imaging system for capturing multi-spectral image data for a cytological specimen, said imaging system comprising:
(a) an optical stage having a light source for illuminating the specimen, focusing means for focusing said light source on a selected area of said cytological specimen wherein said cytological specimen comprises a monolayer specimen, and optical means for producing images of the illuminated area of the specimen in a plurality of spectral bands,
(b) an image capture camera having means for simultaneously capturing said spectral images; and
(c) processing means for processing said captured spectral images simultaneously for evaluating the illuminated specimen.

24. The imaging system as claimed in claim 23, wherein said optical means includes a prism assembly, said prism assembly being optically coupled to the output of said light source and having an optical element for each of said spectral images, and said prism assembly including a narrow band optical filter for each of said spectral bands; and
said spectral bands comprise a first optical band centered at 530 nanometers and having a width of approximately 10 nanometers, a second optical band centered at 630 nanometers and having a width of approximately 10 nanometers, and a third optical band centered at 577 nanometers and having a width of approximately 10 nanometers.
